(19) United States
(12) Patent Application Publication
(10) Pub. No.: US 2009/0259180 A1
(43) Pub. Date: Oct. 15, 2009

(54) Injection method using injector with length-adjustable needle and injection apparatus using the same

(76) Inventor: Jong Soo Choi, Seoul (KR)

Correspondence Address: LADAS & PARRY LLP, 26 WEST 61ST STREET, NEW YORK, NY, 10023, US

(21) Appl. No.: 12/082,552

(22) Filed: Apr. 11, 2008

(51) Int. Cl. A61M 5/46; A61M 5/20

(52) U.S. Cl. 604/117; 604/154

(57) ABSTRACT

An injection method using a length-adjustable needle is disclosed, which comprises an adjusting step in which an exposed needle length of a length-adjustable needle of an injector corresponding to a skin tissue of a patient is adjusted; an insertion step in which an injector is positioned on a skin tissue in a vertical direction with respect to the skin tissue, and a needle is inserted; a suction step in which it is checked whether the needle is inserted into a blood vessel; and an injection step in which an injection liquid is injected from the injector inserted into the skin tissue by the depth corresponding to the exposed length of the needle.

### TECHNICAL FIELD

The present invention relates to an injection apparatus with a length-adjustable needle in which an operator can accurately inject an injection liquid at a desired depth in a skin tissue of a patient, and an injection method for vertically injecting an injection liquid into a skin tissue of a patient by using the above injection apparatus.

### BACKGROUND ART

The injection used in hospitals with respect to a human body or an animal can be classified into a muscle injection, a blood vessel injection, an outer skin injection or something. During an operation of an injection by an operator, a standard needle corresponding to a desired operation purpose is used in compliance with the health and sanitation law. So, the hospitals are equipped with a large size needle for a muscle injection, a middle size injection for a blood vessel injection and a small size needle for an outer skin injection. The companies for manufacturing or supplying injection needles supply the needles in a set and packed type.

The typical needle, which can be easily purchased by an ordinary person, includes a cylinder type injector body, a piston which reciprocates in the interior of the body, and a needle hub having a hub engaged at a front end of the body. The large size needle is generally used in the dermatology and dental surgery hospitals so as to inject in a shallow skin tissue. Since the operator should make an accurate injection at a certain depth in the skin, so the surgical operation may make the operator and patient get tired. So, it is almost impossible to repeatedly make injections along with uniform depths of injections. When the patient sees a large size needle, the patient may be afraid of a large size needle before the operation, which leads to an unstable operation.

FIG. 1 is a cross sectional view illustrating an injection apparatus and an injection method in the conventional art. When injection liquid (filler) is injected for a beauty surgical operation such as a plastic surgery or a skin transformation, the injection liquid should be injected between a dermis D and a fat tissue F for a more effective operation. In this case, an operator inserts a needle of an injector into a skin of a patient at a certain inclination angle by using an injector with a long needle, and then injects a certain amount of injection liquid and pulls out the needle by a certain depth and then injects injection liquid again. The above procedures are repeatedly performed, so the injected portions of the injection liquid remain non-uniform. When the injection liquid is injected into the dermis D, the dermis D is widened and swollen, while generating a lot of pain. When the injection liquid is injected into the epidermis E, lumps may develop on the injected portions. As the injection needle is inserted into a skin at an inclination angle and is pulled out while injecting injection liquid several times, the skin may be stimulated and may be hurt, so the patient can have a lot of pain. With the above problems, the plastic operation or skin transformation surgery may not be normally performed. Even when a relatively normal operation is conducted, it is impossible to obtain continuous effects.

### DISCLOSURE OF THE INVENTION

Accordingly, it is an object of the present invention to provide an injection method using a needle length-adjustable injector and a needle length-adjustable injector which overcome the problems found in the conventional art.

It is another object of the present invention to provide an injection method using a needle length-adjustable injector and a needle length-adjustable injector in which it is possible to inject an injection liquid into a skin by a desired depth, and an injection liquid can be efficiently injected by vertically injecting a needle into a patient's skin by using a length-adjustable needle, which leads to a safer injection.

To achieve the above objects, there is provided an injection method using a length-adjustable needle which comprises an adjusting step in which an exposed needle length of a length-adjustable needle of an injector corresponding to a skin tissue of a patient is adjusted; an insertion step in which an injector is positioned on a skin tissue in a vertical direction with respect to the skin tissue, and a needle is inserted; and an injection step in which an injection liquid is injected from the injector inserted into the skin tissue by the depth corresponding to the exposed length of the needle.

### EFFECTS OF THE INVENTION

In the present invention, a uniform depth injection can be obtained by using a length-adjustable needle, and an injection efficiency and an injection safety can be obtained by injecting into a skin in a vertical direction, and a patient has less pain.

### BRIEF DESCRIPTION OF THE DRAWINGS

The present invention will become better understood with reference to the accompanying drawings which are given only by way of illustration and thus are not limitative of the present invention, wherein;

FIG. 1 is a cross sectional view illustrating a conventional injection method;

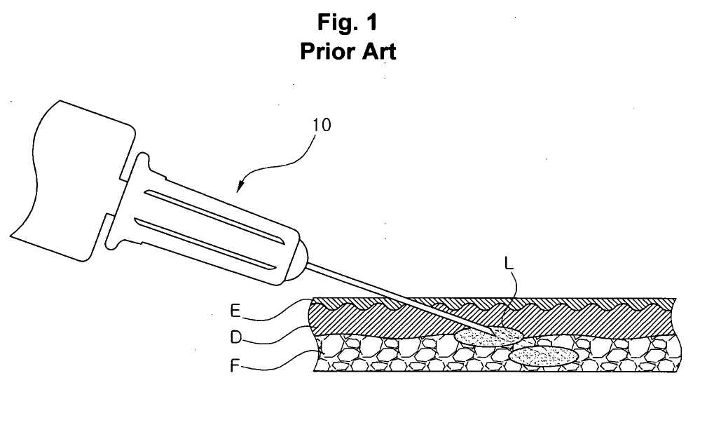

FIG. 2 is a perspective view illustrating a procedure for adjusting an exposed length of a needle by using an adjusting member in an injector according to the present invention;

FIG. 3 is a cross sectional view illustrating a procedure for inserting a length-adjusted needle injector into a skin of a patient according to the present invention;

FIG. 4 is a cross sectional view illustrating a procedure for pressurizing an injected portion by using a depressor after an injection liquid is injected by using an injector according to the present invention;

FIG. 5 is a disassembled perspective view illustrating the whole construction of an injector according to the present invention;

FIG. 6 is a disassembled perspective view illustrating the detailed construction of an injector according to a first embodiment of the present invention;

FIG. 7 is an engaged cross sectional view of an injector of FIG. 6;

FIG. 8 is a disassembled perspective view illustrating the detailed construction of an injector according to a second embodiment of the present invention;

FIG. 9 is an engaged cross sectional view illustrating an injector of FIG. 8;

FIG. 10 is a disassembled perspective view illustrating the detailed construction of an injector according to a third embodiment of the present invention;

FIG. 11 is a disassembled perspective view illustrating the construction of an injector equipped with a multiple needle according to a fourth embodiment of the present invention;

FIG. 12 is a cross sectional view illustrating an injector of FIG. 11;

FIG. 13 is a cross sectional view illustrating a procedure for performing an insertion step by using a multiple needle of FIG. 11;

FIG. 14 is a perspective view illustrating an apparatus which is able to automatically inject an injection liquid of an injector according to the present invention;

FIG. 15 is a cross sectional view illustrating a procedure for performing an insertion step by using the apparatus of FIG. 14; and

FIG. 16 is a schematic view illustrating an automatic injection apparatus.

### MODES FOR CARRYING OUT THE INVENTION

The injection method using a length-adjustable needle according to the present invention will be described with reference to the accompanying drawings.

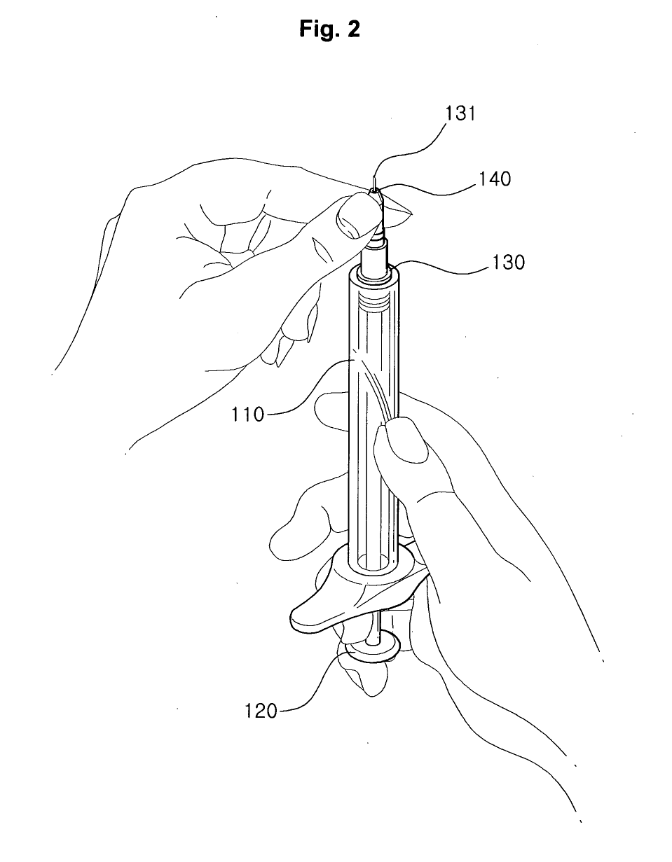

The operation method of an injector having a length-adjustable needle 31 will be described with reference to FIGS. 2 through 4.

The above operation method comprises an adjusting step in which an operator adjusts the length of a needle 131 by using an adjusting member 140 with respect to a skin which will be injected. Here, the construction of the injector adapted to the injection method of the present invention will be described later.

For example, when an ordinary type injector is used, an operator needs to engage a needle hub having a needle proper to a skin portion, which will be injected, to an injector body, but in the present invention the operator needs to engage a needle hub having only one kind of a needle irrespective of its length to the injector body 110, so that various types of injections can be performed. Even when an injector having a small size needle is needed, the exposed length of the needle can be adjusted by rotating the adjusting member 140 in a state that a middle size or a large size needle is engaged.

The adjusting member 140 helps the costs needed for the manufacture and supply of the needles go down as compared to the conventional art in which the needle 131 is made in various sizes. In the conventional art, the metallic needle 131 is integral with the needle hub 130, which is generally made of a synthetic resin material. In this case, the needle 131 should be insert-injected with respect to the needle hub 130 during the manufacture of the above needle. In the manufacture of a small size needle, a more accurate manufacture process is required as compared to a middle size or a large size needle, which leads to a more accurate manufacture facility, so that various manufacture lines are needed for thereby increasing the costs. However, in the present invention, the adjusting member 140 can be separately manufactured by an injection molding method depending on the size of the needle hub 130. So, it should be appreciated that the costs needed for a design change and a purchase of a manufacture facility for implementing various sizes of needles can be significantly saved.

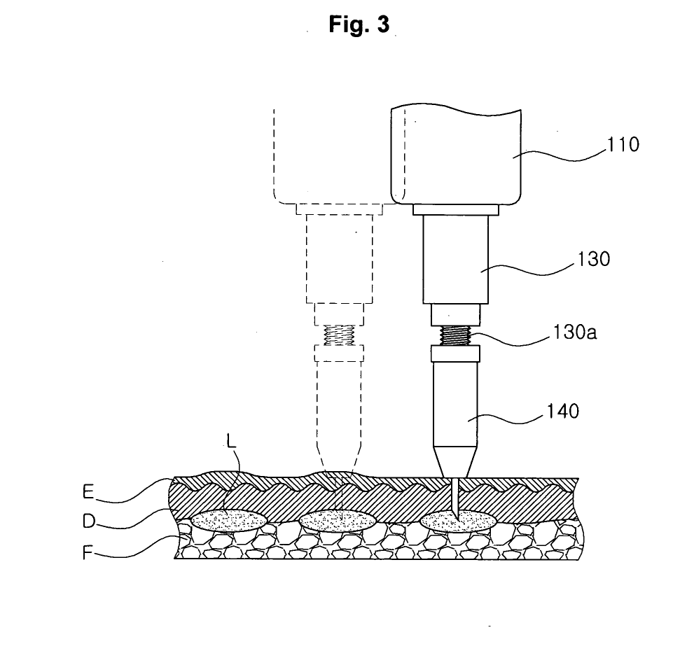

As shown in FIG. 3, the injection method of the present invention comprises an insertion step in which the injector is positioned vertically with respect to a skin tissue, and then the needle 131 is inserted into the skin. Since the operator can easily adjust the exposed length of the needle 131 by using the adjusting member 140, it should be appreciated that the medical accidents, which may occur when the needle 131 not proper for a desired injection depth is used, can be significantly decreased.

For example, it is most preferred that the injection liquid L is injected into a boundary between the dermis D and the fat tissue F when an injection liquid (filler) is injected for a plastic surgery or a skin transformation. In the above adjusting step, when the exposed length of the needle 131 is set by rotating the adjusting member 140 engaged to the needle hub 130 in one direction, the exposed needle 131 can be shortened as compared to the original needle length. When the exposed length-adjusted needle 131 is inserted into the skin tissue in a vertical direction, the tip of the needle 131 through which the injection liquid L is discharged can be correctly positioned between the dermis D and the fat tissue F. When the needle is inserted into the skin in a vertical direction, an insertion can be very accurately adjusted, so the injected skin bleeds least. Since the patient is not afraid of a shorter needle 131, the operator can operate more easily.

As shown in FIG. 3, the injection step of the present invention includes an injection step in which the injection liquid can be accurately injected from the injector into a desired depth portion.

Even when the operator inserts the needle 131 into a skin of the patient a few times in a state that the length of the needle 131 is adjusted counting on the depth between the dermis D and the fat tissue F of the patient, the injection liquid can be uniformly injected into between the dermis D and the fat tissue F.

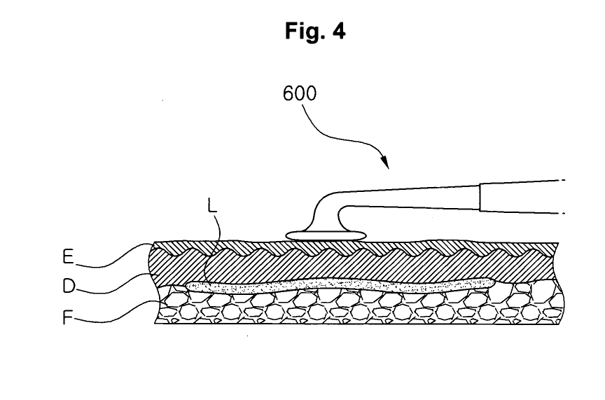

The injection method of the present invention further includes a pressurization step in which an injection portion is pressurized. In the above pressurization step, a depressor 600 of FIG. 4 is adapted. The depressor 600 has a straight shape or bent shape body, with a pressurizing part being formed at one end or both ends of the body in an inwardly concave shape. The injected skin portions generally have irregular concave or convex shapes after the injection, so the operator is needed to massage the injected skin portions to make the uneven surfaces flat and smooth, otherwise bruises develop in the injected skin portions. In the present invention, the injected skin portions can be pressurized and massaged by using the concave pressurization parts of the depressor 600, which leads to overcoming the conventional problems.

When the injections are performed by using the injector with the length-adjustable needle according to the present invention, it should be appreciated that many advantages are obtained. The following table 1 shows the advantages of the present invention as compared to the conventional art counting on a result of the injections obtained after the injection is performed by using the injector with a length-adjustable needle according to the present invention and after the injection is performed as an injection liquid is inserted at an inclined angle by using a conventional needle.

TABLE 1

| | Present invention | Conventional art |
|---|---|---|
| Bleeding | Less | More |
| Pain | Painless | Pain full |
| Tissue damage | Small | More |
| Ecchymosis | Less | More |
| Lump | Less | More |
| Needle/technical | evenly injection method | long length needle |
| (gauge, length) | exactly skin thickness | difficulty (high pressure) |
| | Easy (low pressure | |
| Patient satisfaction | Very good | Big complaint |
| Duration (years) | Long term | Short term |

As seen in the above table 1, a painless injection can be obtained in the injection method in which the injection liquid is injected by using an injector with a length-adjustable needle according to the present invention, and a long injection effect can obtained, and the patient's satisfaction is high.

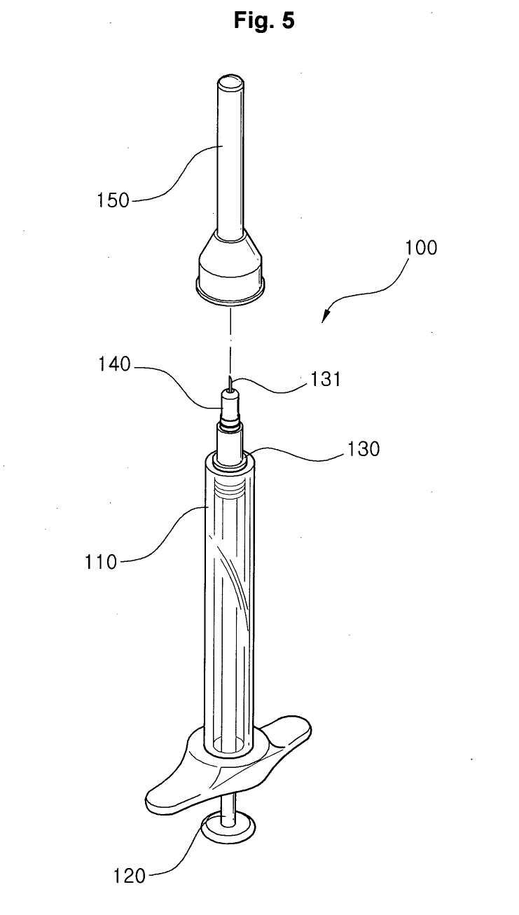

FIG. 5 is a view illustrating the entire constructions of the injector for implementing the injection method of the present invention.

The injector for implementing the injection method of the present invention comprises a cylindrical injector body 110 having a certain storing space for storing an injection liquid therein, and a piston 120 which is inserted from a rear end of the body 110 for forwardly pushing an injection liquid.

A needle hub 130 is engaged to a forward portion of the body 110, and a needle 131 is protruded from a front end of the needle hub 130 by a certain length. An adjusting member 140 is disposed at an outer side of the needle hub 130 for adjusting the length of the needle.

A protection cap 150 is covered on a front side of the body 110, so the needle 131 is not exposed to the outside after the injector is manufactured. The protection cap 150 is able to prevent the damage of the needle 131 and a certain accident which may occur due to the needle 131.

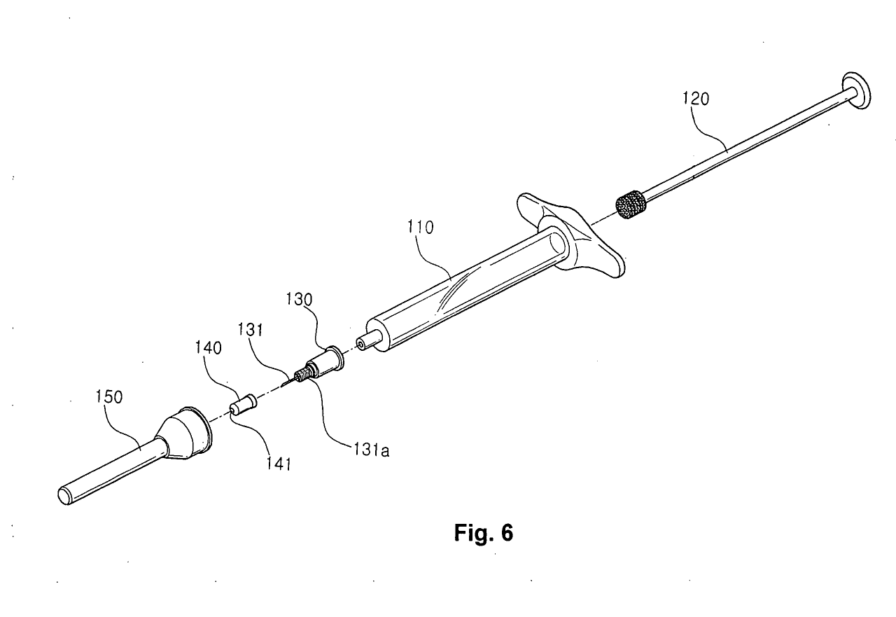

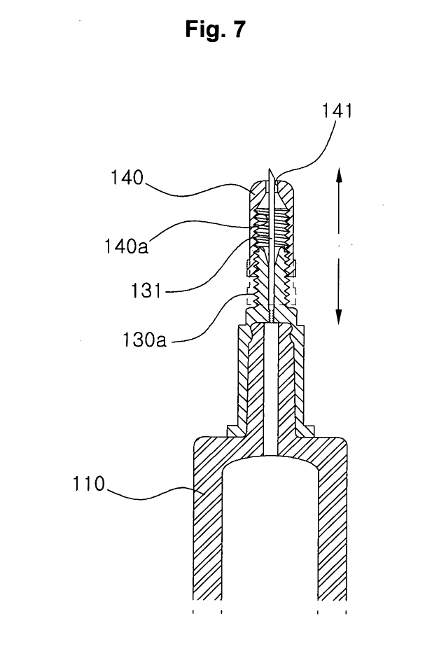

FIGS. 6 and 7 are views illustrating the entire constructions of the injector according to a first embodiment of the present invention.

As shown therein, the piston 120 is inserted into the injector body 110 from its rear end, and the needle hub 130 having a detachable needle 131 is engaged to the front side of the body 110. The above construction of the present invention is a little similar with the conventional injector.

However, in the injector of the present invention, when it is needed to adjust the exposed length of the needle 131 depending on a surrounding situation, the exposed length of the needle 131 can be adjusted by using the adjusting member 140 disposed at an outer side of the needle hub 130. The adjusting member 140 is made of a hollow cylindrical shape, and a through hole 141 is formed in its upper center portion, so the needle 131 passes through the through hole 141.

A thread 130a is formed on a rear outer surface of the needle hub 130, and a thread 140a corresponding to the thread 130a is formed on a rear inner surface of the adjusting member 140.

Here, the inner diameter of the adjusting member 140 corresponds to the outer diameter of the needle hub 130, and the outer diameter portion of the needle hub 130 is accommodated in the interior of the protection cap 150. So, in the present invention, it is not needed to change the construction of the conventional injector or needle 131 for an application of the adjusting member 140.

The adjusting member 140 is engaged at an outer side of the needle hub 130 for the reasons that the length of the needle 131 exposed from a front end of the injector by means of the adjusting member 140 can be adjusted. Here, the unit cost of the adjusting member 140 is much smaller as compared to the needle hub 130 having the needle 131. Here, the needle hub 130 costs too much because it is generally manufactured in many types and is supplied based on the standards. In a state that the needle hub 130 is assembled at a front end of the injector body 110, only part of the needle 131 is exposed toward the front side of the adjusting member 140 for an easier assembling and disassembling work.

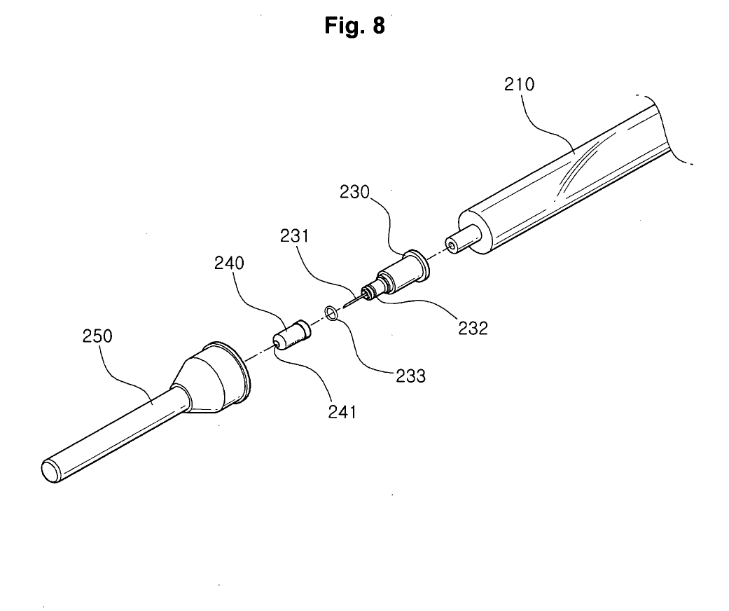

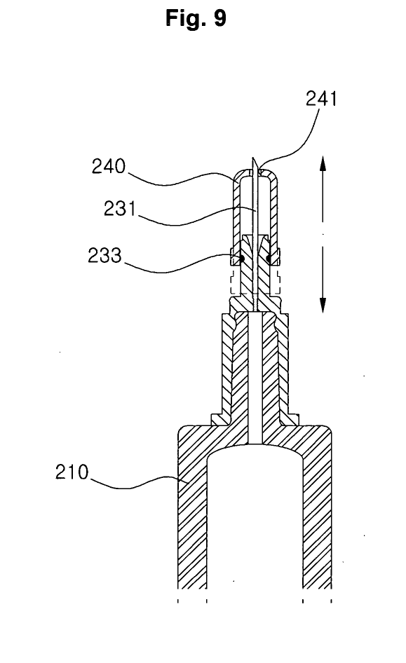

FIGS. 8 and 9 are views illustrating the construction of the injector according to a second embodiment of the present invention.

As shown therein, the construction of the injector of the second embodiment of the present invention is very similar with the construction of the first embodiment of the present invention. The injector according to the second embodiment of the present invention comprises a cylindrical injector body 210 having a certain space for storing an injection liquid therein, a piston 220 which is inserted into the body 210 from its read end and pushes forward the injection liquid, a needle hub 230 having a needle 231 engaged at a front end of the body 210, and an adjusting member 240 which is engaged to an outer side of the needle hub 230.

In the first embodiment of the present invention, the threads are formed on the outer surface of the needle hub 130 and the inner surface of the adjusting member 140, respectively, in a matching structure. But, in this embodiment, a mounting groove 232 is formed on an outer surface of the needle hub 230 in a surrounding direction, and an O-ring 233 is engaged to the mounting groove 232. Since the O-ring 233 contacts with the inner surface of the adjusting member 240, it is preferably made of a rubber material for preventing the scratches or damages of the adjusting member 240 and reliably fixing the adjusting member 240 in place.

The exposed length of the needle 231 can be adjusted by pressurizing the adjusting member 240 in the forward or backward direction in a state that the inner surface of the adjusting member 240 contacts with the O-ring 233.

In this embodiment of the present invention, since only a process for forming the mounting groove 232 in the needle hub 230 is needed without performing a tapping work so as to form the threads in the needle hub 230 and the adjusting member 240, the manufacturing procedures and the manufacturing cost can be significantly decreased in the present invention.

In the second embodiment of the present invention, the O-ring 233 is disposed, but a certain contact protrusion (not shown) made of a rubber material can be formed on an outer surface of the needle hub 230 instead the O-ring 233.

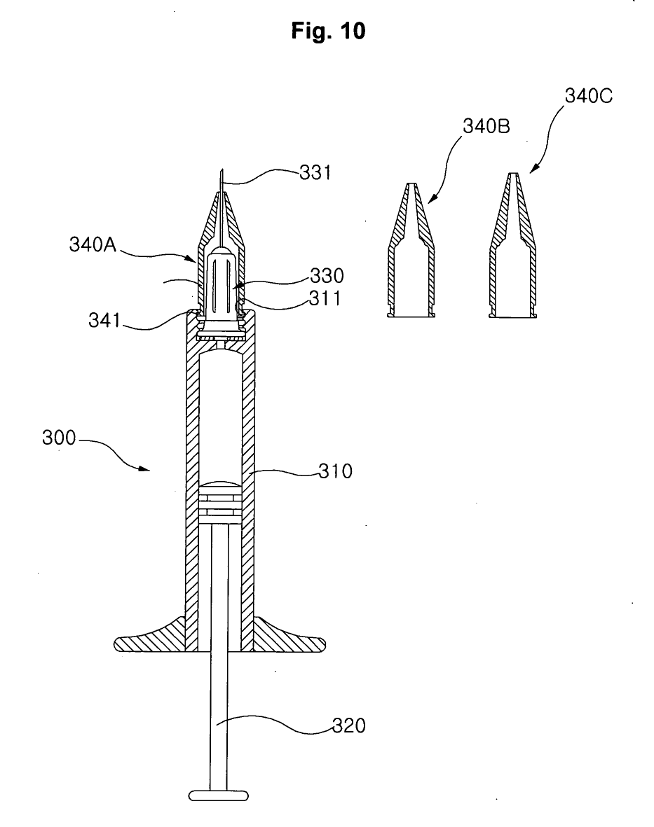

FIG. 10 is a view illustrating the construction of the injector according to a third embodiment of the present invention.

Even in case that a large size needle is engaged to the injector body 310, the adjusting members 340a, 340b and 340c can be adapted for adjusting the lengths of the needles. In the present invention, it is not needed to change the shapes of the injector and the needle for applying the adjusting members 340a, 340b and 340c.

The outer diameter of the lower end of each of the adjusting members 340a, 340b and 340c is determined, so it can be assembled to the inner diameter of the engaging part 311 of the injector body 310.

A locker 341 engaged to the inner side of the engaging part 12 by a thread engagement method is protruded from a lower side of each of the adjusting members 30a, 30b and 30c.

The adjusting members 340a, 340b and 340c are made with various sizes for the different injection depths of the needles, so a proper one can be selected among the adjusting members 340a, 340b and 340c for a corresponding injection and is engaged to the engaging part of the injector.

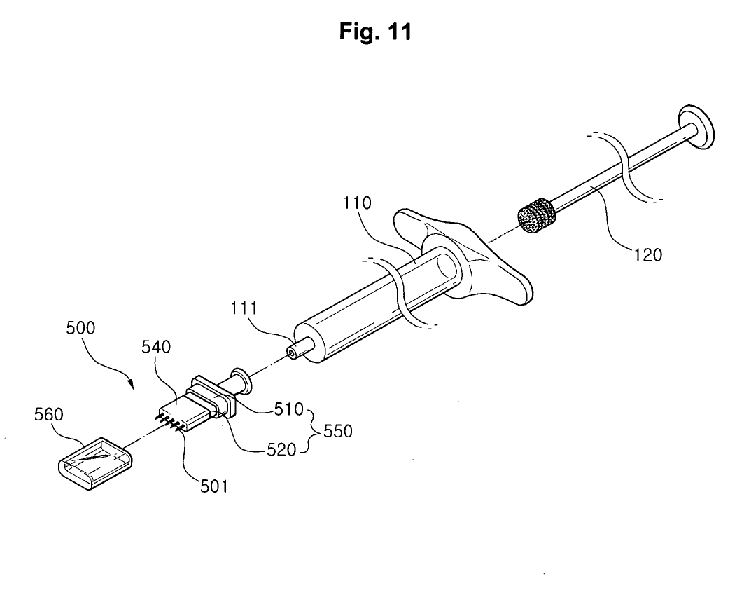

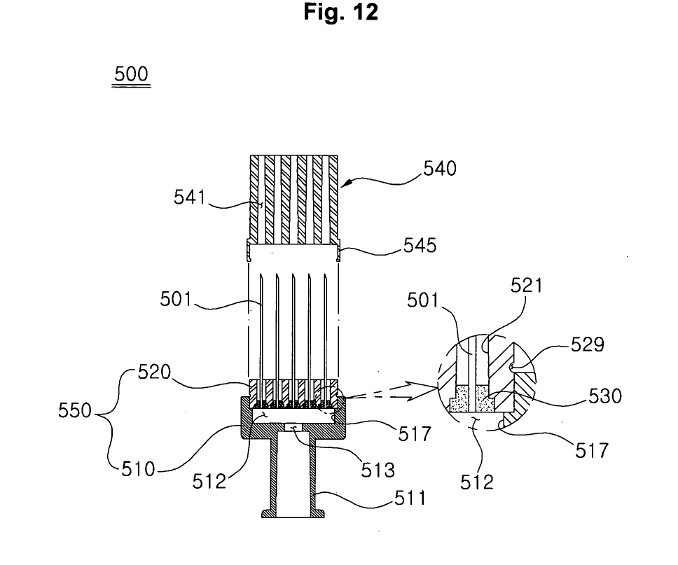

As shown in FIGS. 11 through 13, in the insertion and injection steps of the injection method of the present invention, a multiple needle 500 can be used with multiple needles being arranged in at least one row or column. The multiple needle 500 according to the present invention is equipped with multiple needles as compared to the common injection which is equipped with one needle, so that multiple injections can be performed with one time injection.

The multiple needle 500 is connected to a nozzle 111 protruded from a front end of the injector 110. The multiple needle 500 comprises one main hub 510 engaged at the nozzle 111, and a sub-hub 520 having a plurality of needles 501 engaged at the main hub 510. The adjusting member 540 is provided at an upper side of the needle hub 550 for adjusting the exposed lengths of the needles depending on the insertion depths of the needles for a corresponding operation. The adjusting members 540 having different lengths can be used for changing the exposed lengths of the needles 501.

One inlet 511 and a plurality of outlets 521 are provided in the main hub 510. The inlet 511 of the main hub 510 is protruded, and the nozzle 111 of the injector 110 is inserted into the inner side of the same. The outlet 521 of the main hub 510 communicates with the inlet 511 inside the main hub 510. The injection liquid inputted into the inner side of the main hub 510 through the inlet 511 is discharged to the outside of the main hub 510 through a plurality of the outlets 521. A plurality of needles 501 are provided, each needle being inserted into a corresponding outlet 521, respectively. The needle 501 can be fixedly inserted into the outlet 521, and a sealant fixture 530 made of a certain material such as silicon may be filled between the outlet 521 and the needle 501.

In the main hub 510 according to the present invention, the outlets 521 and the needles 501 can be arranged in a row, so it is possible to uniformly inject in wider areas at one time. The above construction is not limited thereto. In another embodiment of the present invention, the outlets 521 and the needles 501 may be arrange din a plurality of rows or a plurality of columns, so wider areas can be injected at one time.

The main hub 510 is connected to the injector 110 with an inlet 511 being formed at its one end, and an accommodating space 512 is formed at the other end of the same. The inlet 511 is formed in a cylindrical shape which is protruded from one end of the main hub 510 to the outside. The accommodating space 512 is formed in a concave shape that it is concaved from the other end of the main hub 510 to an inward direction. A step 513 is formed at a bottom edge portion of the accommodating space 512, and the inlet 511 and the accommodating space 512 formed in the main hub 510 communicate with each other.

A plurality of outlets 521 pass through the sub-hub 520, and a plurality of needles 501 are disposed in each outlet 521. Each needle 501 may be fixedly inserted into each outlet 521, and may be fixed by using the sealant fixture 530. Here, the sub-hub 520 is fixedly inserted into the main hub 510.

The sub-hub 520 is fixedly inserted into the accommodating space 512 of the main hub 510 with its lower end being mounted on the step 513. So, a certain space is formed between the bottom of the accommodating space 512 and the lower end of the sub-hub 520 as high as the height of the step 513. The inlet 511 and the outlet 521 communicate with each other through the space so formed.

The second body 520 is formed shorter and is fixedly inserted into the accommodating space 512 of the first body 510, and the adjusting member 540, which limits the insertion depth of the needle 501, is detachably engaged to the second body 520.

For example, a groove 529 is formed on an outer surface of the second body 520, and a hook 545 inserted into the groove 529 is formed at a lower end of the adjusting member 540. A plurality of through holes 541 re formed in the adjusting member 540, and each through hole 541 is formed opposite to the outlet 521 of the second body 520. When the adjusting member 540 is mounted in the second body 520 of the needle hub 550, each needle 501 disposed in a corresponding outlet 521 passes through a corresponding through hole 541 of the adjusting member 540, and the tip of each needle 501 is exposed to the outside. The length of the exposed tip of the needle 501 exposed after it passes through the adjusting member 540 is long enough for the exposed tip of the needle to insert up to the boundary of the dermis D and the fat tissue F through the epidermis E for injecting a beauty liquid. The protection cap 560 is provided for preventing the damages of the multi needle 500.

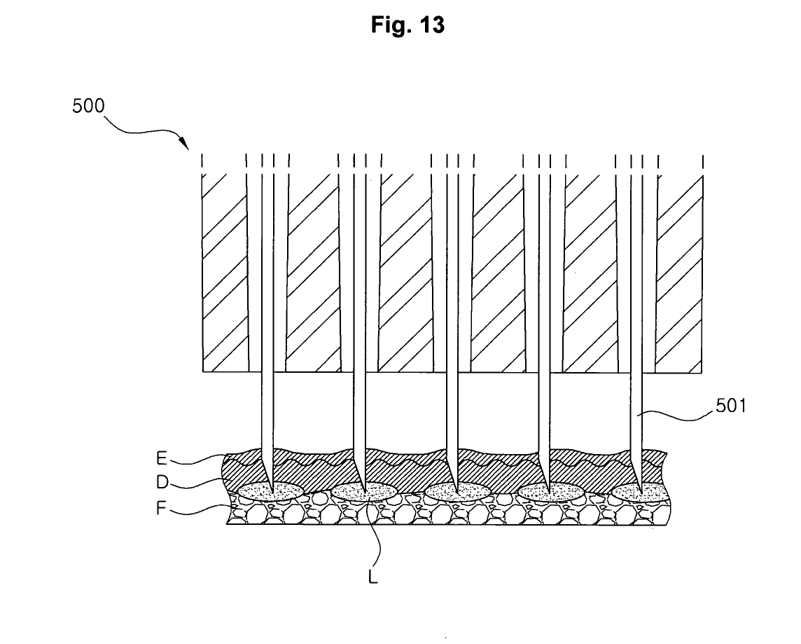

As shown in FIG. 13, it is possible to accurately insert each needle 501 with the simple process that the needle 501 is inserted until the end of the needle hub 550 contacts with the skin, so that multiple injection effects can be obtained with one time injection.

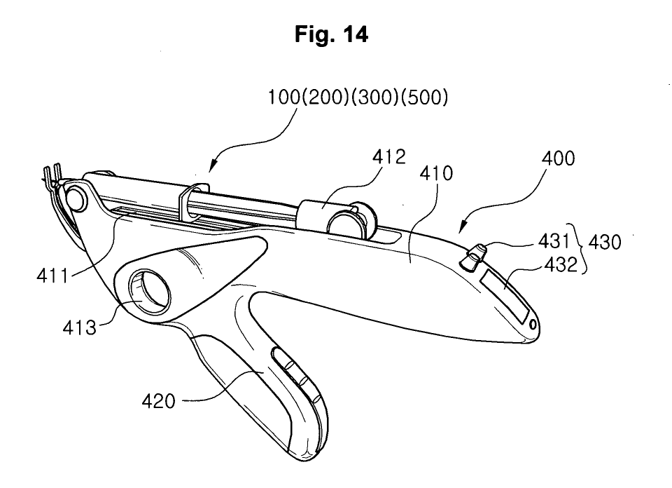

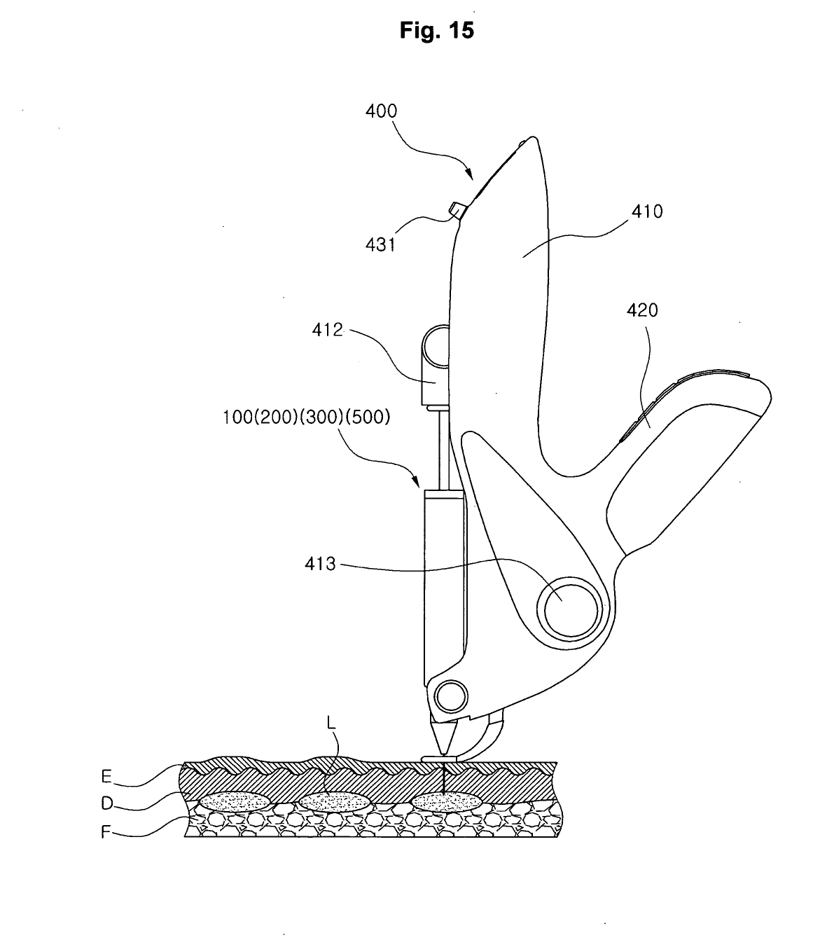

The insertion and injection steps of the injection method of the present invention can be applied to the automatic injection apparatus of FIGS. 14 and 15.

The automatic injection apparatus 400 according to the present invention comprises a body 410 having a handle, an injector transportation unit for transporting the injector, and a piston transportation unit for transporting the piston. A conventional injector may be engaged in the automatic injection apparatus 400, but the injectors 200, 300 and 500 according to the present invention can be preferably engaged.

In more detail, the body 410 is formed in a piston shape with an injector support 411 being movably mounted on the upper side of the body 410, and an operation switch 413 is provided at one side of the handle 420. The body 410 comprises an injector transportation unit 460 which is installed in the interior of the body and is engaged to the injector support 411 and reciprocates the injector support 411 by using a step motor 462, an injector transportation unit 460 in which the piston transportation unit 440 is detachably engaged at the handle, with the piston transportation unit 440 being engaged to the piston of the injector and injecting an injection liquid from the injector by moving the piston, and a control a display unit 430 which includes a battery is installed on a back surface of the body 410 for supplying power to the piston transportation unit 440 and is used for controlling and displaying the operations of the injector transportation unit 460 and the piston transportation unit 440, respectively. The body 410 forms the frame of the automatic injection apparatus 400 according to the present invention. The body can be formed in a certain shape so that a user can easily grab and use. The injector support 411 is movably disposed on the upper side of the main body 410 with an injector being disposed on the injector support. The operation switch 413 is provided in the handle of the body 410, namely in the trigger portion, for operating and stopping the automatic injection apparatus 400 according to the present invention.

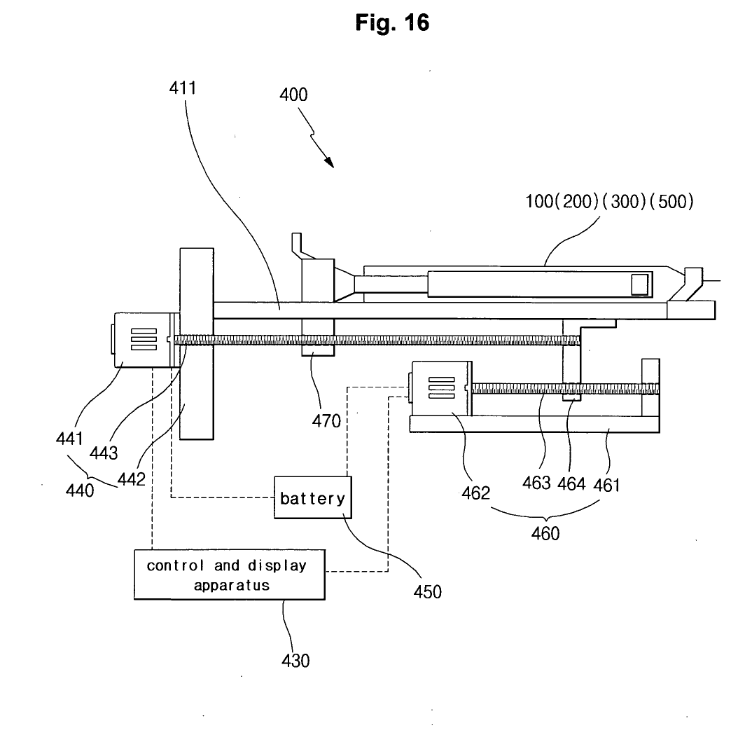

The injector transportation unit 460 is engaged to the injector support 411 in the body 410 for reciprocating the injector support 411. As shown in FIG. 16, the injector transportation unit 460 comprises a first support 461 fixed in the body 410, a first step motor 462 fixed in the first support 411, a first lead screw 463 of which one end is fixed to the shaft of the step motor 462, and the other end is rotatably fixed to the end of the first support 461, and a support holder which moves forward or backward along the first lead screw 463 when the first step motor 462 rotates in normal or reverse directions, and moves the injector support 411 in the forward or backward direction, with one side of the support holder being movably engaged to the first lead screw 463, with the other end of the same being fixed in the injector support 411.

The first support 411 fixed in the body 4110 is bent at a right angle and fixes the first step motor 462 in the horizontal base portion, and the other end of the first lead screw 463 is rotatably engaged to a lateral side that is bent at a right angle from the horizontal base portion.

Here, the step motor 462 fixed in the first support 411 rotates in the normal or reverse direction in accordance with a pulse type control signal of the control and display unit 430 for thereby rotating the first lead screw 463. The step motor (generally referred to stepping motor, stepper motor, pulse motor or the like) is a converter for converting a certain force into a mechanical axial motion in accordance with a pulse type control signal from the control and display unit 430. The shaft of the step motor rotates at a set angle in accordance with the number of pulses, and when it is adjusted depending on the interval of pulses, it is possible to control a driving type and speed proper to the control destination. The first lead screw 463 directly connected to the first step motor 462 converts the rotation motion of the step motor 462 into a straight linear motion and is rotatably disposed between the first step motor 462 and the first support 461.

The support holder 464 movably engaged to the first lead screw 463 and fixed to the injector support 411 moves forward and backward along the first lead screw 463 as the step motor 462 rotates in the normal or reverse direction for thereby moving the injector support 411 in the forward or backward direction.

The piston transportation unit 440 is installed in the body 410, and the piston transportation unit 440 is engaged to the piston of the injector for injecting an injection liquid from the injector into a skin or a body by moving the piston as the injector support 411 moves by means of the injector transportation unit 460. The piston transportation unit 440 comprises a second support 442 integrally formed at a rear end of the injector support 411, a second step motor 441 fixed at a rear side of the second support 442, a second lead screw 443 which is fixed to the shaft of the second step motor 441 and is rotatably fixed to the second support 442 and the support holder 464, respectively, a second step motor 441 of which one side is movably engaged to the second lead screw 443, and the other side is engaged to a rear end of the piston, and a piston holder 412 which moves forward or backward along the second lead screw 443 as the second step motor 441 rotates in normal or reverse direction for thereby moving the piston forward or backward.

The second support 442 integrally formed in a rear end of the injector support 411 fixedly supports the second step motor 441, and one side of the second lead screw 463 is rotatably engaged to the second support 442.

The second step motor 441 fixed at a rear side of the second support 442 rotates in normal or reverse direction at a certain angle in accordance with a pulse type signal of the control and display unit and rotates the second lead screw 443.

The second lead screw 443 directly engaged to the shaft of the second step motor 441 converts the rotational motion of the step motor 441 into a straight reciprocating motion, and is rotatably disposed between the second step motor 441, the second support 442 and the support holder 464, respectively.

The piston holder 412, which is movably engaged to the second lead screw 443 and is engaged to the rear end of the piston, moves forward or backward along the second lead screw 443 as the second step motor 441 rotates in normal or reverse direction for thereby moving the piston in forward or backward direction.

The battery 450 is detachably engaged to the handle of the body 410. The battery 450 is provided for supplying power to the injector transportation unit 460 and the piston transportation unit 440, respectively, and is preferably made of a small lithium battery. In another embodiment, the battery may be formed of chargeable batteries.

The control and display unit 430 is installed in the backside of the body 410. The control and display unit 430 controls and displays the operations of the injector transportation unit 460 and the piston transportation unit 440 and comprises an adjusting dial 431, a control panel which controls the operations of the injector transportation unit 460 and the piston transportation unit 440 for a continuous operation mode depending on the rotation angle of the adjusting dial 431, with the operation mode being formed of the number of injections corresponding to the rotation angle of the adjusting dial 431, an injection liquid amount per injection and an injection depth, and a display panel 431 which displays the operation mode based on the control of the control panel and the volume of the injector and the charging level of the battery. The adjusting dial 431 generates a control signal depending on the rotational angle, and allows the injector transportation unit 460 and the piston transportation unit 400 to operate in a certain operation mode, respectively.

The operation of the automatic injection apparatus 400 will be described. A proper operation mode, number of injections, injection liquid amount per injection, and injection depth are determined by properly operating the adjusting dial 431. The operator holds the body 410, and the front end of the body 410 is contacted with a portion, which will be injected, and the operation switch 413 is pushed, so that the injector transportation unit 460 and the piston transportation unit 440 operate in accordance with a control of the control panel.

In the application of the automatic injection apparatus 400 in the injection method according to the present invention, it is possible to accurately adjust the length of a needle depending on the depth of a skin by engaging the injector with an injection liquid therein, and a lot of skin areas can be automatically injected during a shorter time, and the injection time can be significantly decreased, so the operator and the patient can get less fatigue.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. In an injector which includes an injector body storing an injection liquid therein, and a piston engaged to a rear end of the body for pressurizing the injection liquid, an injection method using a length-adjustable needle, comprising:
an adjusting step in which an exposed needle length of a length-adjustable needle of an injector corresponding to a skin tissue of a patient is adjusted by using a length-adjustable needle injector which comprises a needle hub engaged to a front side of the body and has a needle at its front end, and an adjusting member which is movably engaged to an outer side of the needle hub for thereby adjusting an exposed length of the needle;
an insertion step in which an injector is positioned on a skin tissue in a vertical direction with respect to the skin tissue, and a needle is inserted; and
an injection step in which an injection liquid is injected from the injector inserted into the skin tissue by the depth corresponding to the exposed length of the needle.

2. The method of claim 1, wherein a thread is formed on an outer surface of the needle hub, and an exposed length of the needle is adjusted by using the adjusting member having a thread formed on its inner surface, which corresponds to the thread formed in the needle hub.

3. The method of claim 1, wherein a mounting groove is formed on an outer surface of the needle hub, and an O-ring contacting with an inner surface of the adjusting member is disposed in the mounting groove.

4. In an injector which includes an injector body storing an injection liquid therein, and a piston engaged to a rear end of the body for pressurizing the injection liquid, an injection method using a length-adjustable needle, comprising:
an adjusting step in which an exposed needle length of a length-adjustable needle of an injector corresponding to a skin tissue of a patient is adjusted by using a length-adjustable needle injector which comprises an adjusting member engaged to a front end of the body of the injector and including a cylindrical body, and an assembling unit for engaging an injector to a lower portion of the body, with the inner diameter portion of the assembling unit being inserted into a diameter expansion portion between the needle holder and the assembly protrusion of the injection needle;
an insertion step in which an injector is positioned on a skin tissue in a vertical direction with respect to the skin tissue, and a needle is inserted; and
an injection step in which an injection liquid is injected from the injector inserted into the skin tissue by the depth corresponding to the exposed length of the needle.

5. In an injector which includes an injector body storing an injection liquid therein, and a piston engaged to a rear end of the body for pressurizing the injection liquid, an injection method using a length-adjustable needle, comprising:
an adjusting step in which an exposed needle length of a length-adjustable needle of an injector corresponding to a skin tissue of a patient is adjusted by using a length-adjustable needle injector which comprises a needle hub which is engaged to a front side of the body and includes one inlet into which a nozzle of the injector is inserted, and a plurality of outlets communicating with the inlet, respectively, a plurality of needles which are inserted into each outlet of the needle hub, respectively, and are arranged in at least one row or column, and an adjusting member which is inserted into the needle hub and is able to adjust the length of each needle;
an insertion step in which an injector is positioned on a skin tissue in a vertical direction with respect to the skin tissue, and a needle is inserted; and
an injection step in which an injection liquid is injected from the injector inserted into the skin tissue by the depth corresponding to the exposed length of the needle.

6. In an injector which includes an injector body storing an injection liquid therein, and a piston engaged to a rear end of the body for pressurizing the injection liquid and is used in the injection of claim 1, a length-adjustable needle injector, comprising:
a needle hub which is engaged to a front side of the body with the needle being engaged at a front side of the needle hub; and
an adjusting member which is movably engaged to an outer side of the needle hub for adjusting the exposed length of the needle.

7. In an injector which includes an injector body storing an injection liquid therein, and a piston engaged to a rear end of the body for pressurizing the injection liquid and is used in the injection of claim 4, a length-adjustable needle injector, comprising:
an adjusting member engaged to a front end of the body of the injector and including a cylindrical body, and an assembling unit for engaging an injector to a lower portion of the body, with the inner diameter portion of the assembling unit being inserted into a diameter expansion portion between the needle holder and the assembly protrusion of the injection needle.

8. In an injector which includes an injector body storing an injection liquid therein, and a piston engaged to a rear end of the body for pressurizing the injection liquid and is used in the injection of claim 5, a length-adjustable needle injector, comprising:
a needle hub which is engaged to a front side of the body and includes one inlet into which a nozzle of the injector is inserted, and a plurality of outlets communicating with the inlet, respectively;
a plurality of needles which are inserted into each outlet of the needle hub, respectively, and are arranged in at least one row or column; and
an adjusting member which is inserted into the needle hub and is able to adjust the length of each needle;

9. An injection method using a length-adjustable needle injector of claim 6, comprising
an adjusting step in which an exposed needle length of a length-adjustable needle of an injector corresponding to a skin tissue of a patient is adjusted by using an automatic injection apparatus which comprises:
a piston-shaped body in which the length-adjustable needle injector is engaged in a movable injector support, and an operation switch is disposed at one side of the handle, and a connector connection unit is provided at the other side of the handle;
an injector transportation means which is installed in the body and is engaged to the injector support and reciprocates the injector support by using a step motor;
a piston transportation means which is installed in the body and is engaged to the piston of the injector and moves the piston for thereby injecting an injection liquid from the injector;
a battery which is detachably engaged in the portion of the handle of the body and supplies power to the injector transportation means and the piston transportation means, respectively; and
a control and display means which is installed on a backside of the body and controls and displays the operations of the injector transportation means the piston transportation means and is able to be upgraded with respect to the operation modes of the injector transportation means and the piston transportation means through the connector connection unit, respectively;
an insertion step in which an injector is positioned on a skin tissue in a vertical direction with respect to the skin tissue, and a needle is inserted; and
an injection step in which an injection liquid is injected from the injector inserted into the skin tissue by the depth corresponding to the exposed length of the needle.

10. An injection method using a length-adjustable needle injector of claim 7, comprising
an adjusting step in which an exposed needle length of a length-adjustable needle of an injector corresponding to a skin tissue of a patient is adjusted by using an automatic injection apparatus which comprises:
a piston-shaped body in which the length-adjustable needle injector is engaged in a movable injector support, and an operation switch is disposed at one side of the handle, and a connector connection unit is provided at the other side of the handle;
an injector transportation means which is installed in the body and is engaged to the injector support and reciprocates the injector support by using a step motor;
a piston transportation means which is installed in the body and is engaged to the piston of the injector and moves the piston for thereby injecting an injection liquid from the injector;
a battery which is detachably engaged in the portion of the handle of the body and supplies power to the injector transportation means and the piston transportation means, respectively; and
a control and display means which is installed on a backside of the body and controls and displays the operations of the injector transportation means the piston transportation means and is able to be upgraded with respect to the operation modes of the injector transportation means and the piston transportation means through the connector connection unit, respectively;
an insertion step in which an injector is positioned on a skin tissue in a vertical direction with respect to the skin tissue, and a needle is inserted; and
an injection step in which an injection liquid is injected from the injector inserted into the skin tissue by the depth corresponding to the exposed length of the needle.

11. An injection method using a length-adjustable needle injector of claim 8, comprising
an adjusting step in which an exposed needle length of a length-adjustable needle of an injector corresponding to a skin tissue of a patient is adjusted by using an automatic injection apparatus which comprises:
a piston-shaped body in which the length-adjustable needle injector is engaged in a movable injector support, and an operation switch is disposed at one side of the handle, and a connector connection unit is provided at the other side of the handle;
an injector transportation means which is installed in the body and is engaged to the injector support and reciprocates the injector support by using a step motor;
a piston transportation means which is installed in the body and is engaged to the piston of the injector and moves the piston for thereby injecting an injection liquid from the injector;
a battery which is detachably engaged in the portion of the handle of the body and supplies power to the injector transportation means and the piston transportation means, respectively; and
a control and display means which is installed on a backside of the body and controls and displays the operations of the injector transportation means the piston transportation means and is able to be upgraded with respect to the operation modes of the injector transportation means and the piston transportation means through the connector connection unit, respectively;
an insertion step in which an injector is positioned on a skin tissue in a vertical direction with respect to the skin tissue, and a needle is inserted; and
an injection step in which an injection liquid is injected from the injector inserted into the skin tissue by the depth corresponding to the exposed length of the needle.
